(19) United States
(12) Patent Application Publication
(10) Pub. No.: US 2021/0171268 A1
(43) Pub. Date: Jun. 10, 2021

(54) PROCESS FOR INFUSING A TERPENE SOLUTION INTO A STORAGE CONTAINER THAT STORES CANNABIS

(71) Applicant: Synergy Research Group, Inc., North Hollywood, CA (US)

(72) Inventor: Mike Schneider, Sherman Oaks, CA (US)

(73) Assignee: Synergy Research Group, Inc., North Hollywood, CA (US)

(21) Appl. No.: 16/708,326

(22) Filed: Dec. 9, 2019

(51) Int. Cl. B65D 81/24; A61K 45/06; A61Q 13/00

(52) U.S. Cl. CPC B65D 81/24; A61K 2800/87; A61Q 13/00; B65D 2203/12; A61K 45/06

(57) ABSTRACT

A process is provided for infusing a cannabis storage container with a terpene composition. The process injects a pre-fabricated aroma emission device with the terpene composition. The pre-fabricated aroma emission device has one or more pores through which an aroma from the terpene composition is emitted. Further, the process positions the pre-fabricated aroma emission device within the cannabis storage container. Additionally, the process seals the cannabis storage container. The pre-fabricated aroma emission device emits the aroma of the terpene composition through the one or more pores without human activation after said sealing of the cannabis storage container. Alternatively, a process for manufacture is provided for manufacture of an aroma emission device.

### BACKGROUND

### 1. Field

This disclosure generally relates to terpenes. More particularly, the disclosure relates to terpene infusion for olfactory use.

### 2. General Background

Terpenes are hydrocarbon compounds found in a variety of plants, some of which are cannabis and some of which are not. In particular, terpenes are recognized for their ability to emit a strong aroma of varying scents, depending upon the particular terpene strain. With respect to cannabis, terpenes emit such scents to deter would-be attackers, such as herbivores, from eating the plant, or attract those who are more interested in eating the attackers, such as carnivores.

Furthermore, many cannabis plant growers tend to grow cannabis in a way that strengthens the potency of tetrahydrocannabinol (“THC”), which typically has the psychoactive effect of producing a sense or euphoria in a human being, the result of THC binding with cannabinoid 1 (“CB1”) receptors in the brain. Cannabis plants with a higher potency of THC than others also tend to have the terpenes that emit a smell typically considered to be a desirable aroma to many users of cannabis.

While being grown, cannabis may lose some, if not all, of the aroma associated with its terpenes. The aroma may be lost, or diminished, based on a variety of factors (e.g., seed selection, sub-optimal growing environment, etc.).

As a result, current apparatuses and processes do not adequately maintain the natural aroma from cannabis after being grown.

### SUMMARY

A process is provided for infusing a cannabis storage container with a terpene composition. The process injects a pre-fabricated aroma emission device with the terpene composition. The pre-fabricated aroma emission device has one or more pores through which an aroma from the terpene composition is emitted. Further, the process positions the pre-fabricated aroma emission device within the cannabis storage container. Additionally, the process seals the cannabis storage container. The pre-fabricated aroma emission device emits the aroma of the terpene composition through the one or more pores without human activation after said sealing of the cannabis storage container.

Alternatively, a process for manufacture is provided for manufacture of an aroma emission device. The process positions a terpene composition within a first portion of the aroma emission device. Furthermore, the process adheres a second portion of the aroma emission device to the first portion to seal the aroma emission device. The aroma emission device has one or more pores through which an aroma from the terpene composition is emitted. Additionally, the aroma emission device is configured to be positioned within a cannabis storage container such that the aroma emission device emits the aroma of the terpene composition through the one or more pores without human activation after sealing of the cannabis storage container.

### BRIEF DESCRIPTION OF THE DRAWINGS

The above-mentioned features of the present disclosure will become more apparent with reference to the following description taken in conjunction with the accompanying drawings wherein like reference numerals denote like elements and in which:

FIG. 1A illustrates an example of a lab environment in which a user performs the terpene infusion process via injection into a pre-fabricated aroma emission device.

FIG. 1B illustrates an example of the lab environment in which the user performs the terpene infusion process via injection into an alternative pre-fabricated aroma emission device.

FIG. 1C illustrates an aroma emission device that may be fabricated such that the terpene composition is positioned within it during fabrication.

FIG. 2 illustrates a machine-operated fabrication configuration to fabricate the aroma emission device, illustrated in FIG. 1C.

FIG. 3 illustrates a process for infusing a cannabis storage container with a terpene composition.

### DETAILED DESCRIPTION

A terpene infusion process is provided for infusing a terpene solution into a storage container storing one or more cannabis materials (e.g., cannabis flowers, leaves, etc.). In one embodiment, the terpene infusion process injects a terpene solution into a prefabricated aroma emission apparatus that is positioned within a cannabis storage container. The aroma emission apparatus may be a single-purpose apparatus, whose sole purpose is to emit the aroma from the terpene solution into the cannabis storage container. Alternatively, the aroma emission apparatus may be a multi-purpose apparatus, which provides other functionality (e.g., humidity control, temperature control, etc.) with respect to the cannabis storage container. In another embodiment, the terpene infusion process places the terpene solution within an aroma emission apparatus, which is not prefabricated. In other words, the terpene infusion process may place the terpene solution within the aroma emission apparatus during fabrication of the aroma emission apparatus. (Placement of the terpene solution may encompass injection, dropping, or various other forms of positioning the terpene solution within a pre-fabricated aroma emission device, or an aroma emission device that is not pre-fabricated.)

The aroma emission apparatus may be selected from a variety of different structures of varying shapes and sizes, and also may be fabricated from a variety of different materials. Furthermore, one or more portions of the aroma emission apparatus may have sufficient porousness to allow the aroma from the terpene solution to be emitted within the cannabis storage container without human activation. In other words, the cannabis storage container may remain sealed to avoid undesirable effects (e.g., contamination, excessive humidity, etc.), while at the same time being infused with the aroma from the terpene solution. As a result, when an end-user wants to use the stored cannabis, he or she may unseal the cannabis storage container and smell the aroma from the terpene solution, which counteracts any undesirable smell from the cannabis flowers. Furthermore, such odor emission prevention, or minimization, is accomplished without sacrificing the potency of THC in the one or more cannabis materials. The aroma emission may also help improve the taste of the one or more cannabis materials during use.

The terpene solution itself is not limited to any particular formula. For instance, the terpene solution may include a terpene blend of various terpene materials. As an example, the terpene materials may include one or more naturally derived non-cannabis terpene isolates, which are food grade. As another example, the terpene materials may include one or more terpenes extracted from one or more cannabis plants. As yet another example, the terpene materials may form a cannabis/non-cannabis hybrid formulation, including one or more terpenes extracted from one or more cannabis plants and one or more naturally derived non-cannabis terpene isolates.

Furthermore, the terpene blend may include materials other than specific terpenes. For instance, the terpene blend may include one or more essential oils. Additionally, or alternatively, the terpene blend may include one or more flavor enhancers, such as an ester and an aldehyde.

Regardless of the particular terpene composition, the terpene infusion process provided for herein may be applied to the cannabis storage container to counteract/modify the olfactory effects naturally emitted from the cannabis stored within the cannabis storage container. In one embodiment, the terpene infusion process utilizes a particular terpene composition, and quantity thereof, that has been determined to meet a predetermined olfactory threshold for cannabis odor prevention/minimization. For example, a baseline odor for cannabis may be established for comparison with cannabis emissions in the cannabis storage container that has been infused with the terpene composition according to the terpene infusion process provided for herein.

An olfactometer may be utilized to measure how much dilution of the baseline cannabis odor emission has occurred after terpene infusion for a predetermined amount of time (e.g., minutes, hours, days, etc.). Accordingly, a particular terpene composition, and quantity therefore, that meets a cannabis odor dilution threshold (e.g., a range of forty percent to one hundred percent dilution) may be established as the terpene composition that is utilized for the terpene infusion process. In addition, or in the alternative, the olfactometer may be utilized to test terpene infused cannabis storage containers prior to shipment. Accordingly, not only may the olfactometer be utilized to select a particular terpene composition, but also for quality control in ensuring that cannabis storage containers are infused with proper amounts of terpenes to dilute odor emission from the stored cannabis according to the cannabis odor dilution threshold. (The olfactometer is just one example of a measurement device for measuring cannabis odor dilution efficacy. Other devices and/or processes may be used instead for such measurements and/or determinations.)

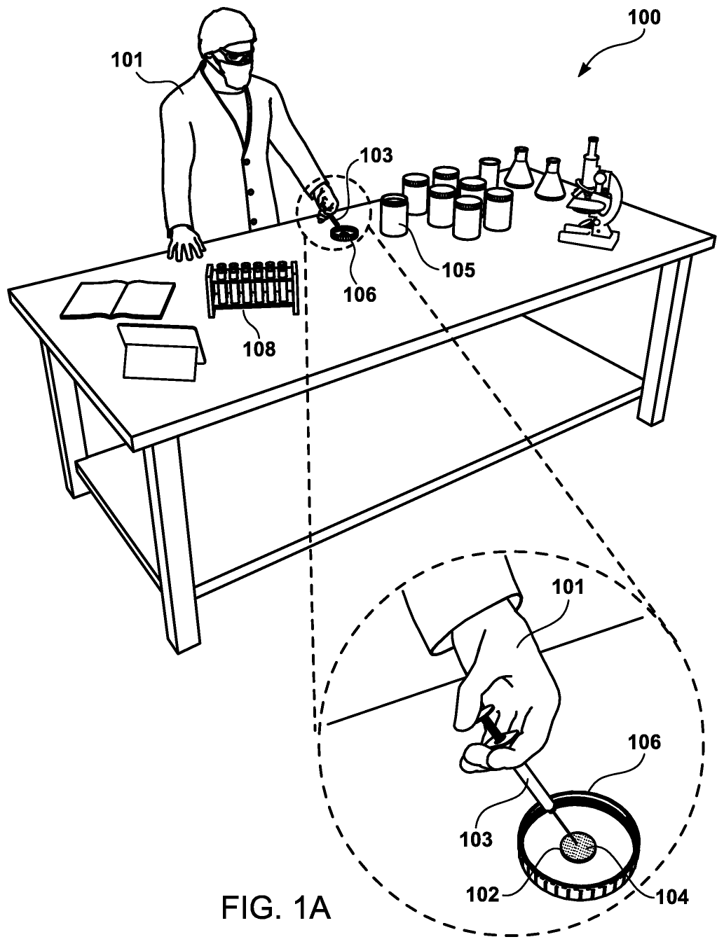

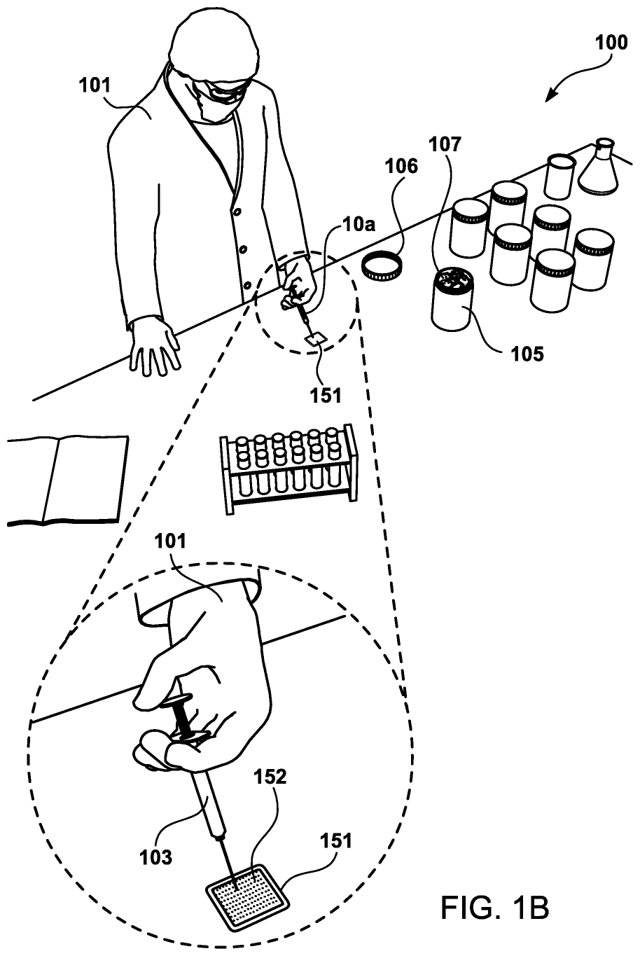

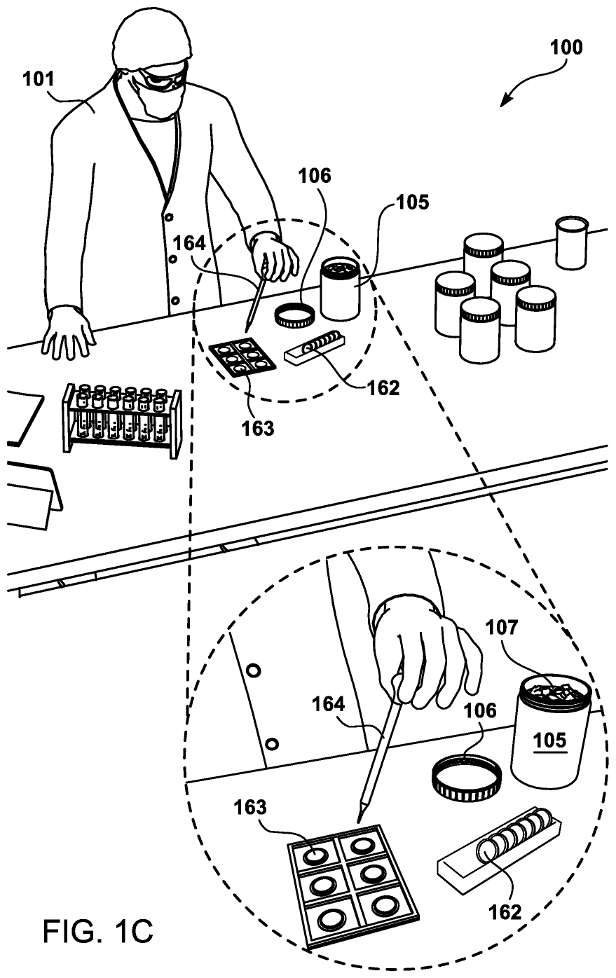

FIGS. 1A-1C illustrate various examples of implementations of the terpene infusion process. In particular, FIG. 1A illustrates an example of a lab environment 100 in which a user 101 performs the terpene infusion process via injection into a pre-fabricated aroma emission device 102. In other words, the user 101 may avoid having to expend resources on manufacturing an aroma emission device, instead opting to focus on injecting the terpene composition into an aroma emission device that has already been manufactured. For instance, the user 101 may inject, via an injection tool 103 (e.g., a syringe), the terpene solution into the pre-fabricated aroma emission device 102. (A syringe is just one example of the injection tool 103. Other types of injection tools 103 may be used instead.) The terpene solution may have been stored in a storage container 108 (e.g., test tube, vial, etc.). In one embodiment, the aroma emission device 102 may have one or more pores for aroma emission, or one or more other emission purposes. The user 101 may position a tip of the injection tool 103 in proximity to, or within, the one or more pores 104 to inject the terpene composition in the aroma emission device 102.

In one embodiment, the pre-fabricated aroma emission device 102 is adhered to a portion of a cannabis storage container 105, which may store cannabis materials 107. As an example, the pre-fabricated aroma emission device 102 may be adhered to an inner portion (e.g., underside) of a cap 106. As another example, the pre-fabricated aroma emission device 102 may be adhered to a wall of the cannabis storage container 105. As yet another example, the pre-fabricated aroma emission device 102 may be adhered to the inside bottom portion of the cannabis storage container 105. In essence, the pre-fabricated aroma emission device 102 may be adhered to an inside portion of the cannabis storage container 105 so that the pre-fabricated aroma emission device 102 emits an aroma during storage of the cannabis materials 107, after the cannabis storage container 105 has been sealed, without human activation after the sealing. In other words, a human operator does not have to activate any type of nozzle, or other emitter, to emit the aroma from the terpene composition.

The pre-fabricated aroma emission device 102 may be adhered to the portion of the cannabis storage container 105 through a variety of adherents (e.g., glue, adhesive, hook-and-loop fastener, clips, magnets, etc.). Alternatively, the pre-fabricated aroma emission device 102 may be inserted into a compartment (e.g., pocket) specifically configured to receive the pre-fabricated aroma emission device 102. In another embodiment, the pre-fabricated aroma emission device 102 may rest inside the cannabis storage container 105 (e.g., on, or in between, the cannabis flowers).

Furthermore, the pre-fabricated aroma emission device 102 may be manufactured according to a variety of different shapes (e.g., cylindrical, cubic, rectangular, circular, etc.) and sizes.

Moreover, FIG. 1B illustrates an example of the lab environment 100 in which the user 101 performs the terpene infusion process via injection into an alternative pre-fabricated aroma emission device 151. For example, the alternative pre-fabricated aroma emission device 151 may be a pouch with one or more pores 152. The pouch may be constructed from one or more interwoven materials (e.g., fibrous materials), in between which rest the one or more pores 152. Alternatively, the pouch may be constructed from other materials (e.g., paper). In one embodiment, the pouch may be adhered to a portion of the cannabis storage container 105. In another embodiment, the pouch may rest inside the cannabis storage container 105 (e.g., on, or in between, the cannabis flowers) without adherence to a portion of the cannabis storage container 105.

Alternatively, as illustrated in FIG. 1C, an aroma emission device may be fabricated such that the terpene composition is positioned within it during fabrication. For example, the aroma emission device may have a first portion 162 (e.g., top) and a second portion 163 (e.g., bottom). The user 101 may use a dropper 164 to obtain the terpene solution from the storage container 108, and emit the terpene solution into the second portion 163. Subsequently, the user 101 may adhere the first portion 162 to the second portion 163 to fabricate the aroma emission device 161. Various adherents (e.g., glue, adhesive, magnets, clips, screws, pins, etc.) may be used to adhere the first portion to the second portion.

Other quantities of portions of the aroma emission device may be used instead of two portions. For example, the aroma emission device may be fabricated as a single unit, without distinct portions. As another example, the aroma emission device may be fabricated from more than two portions.

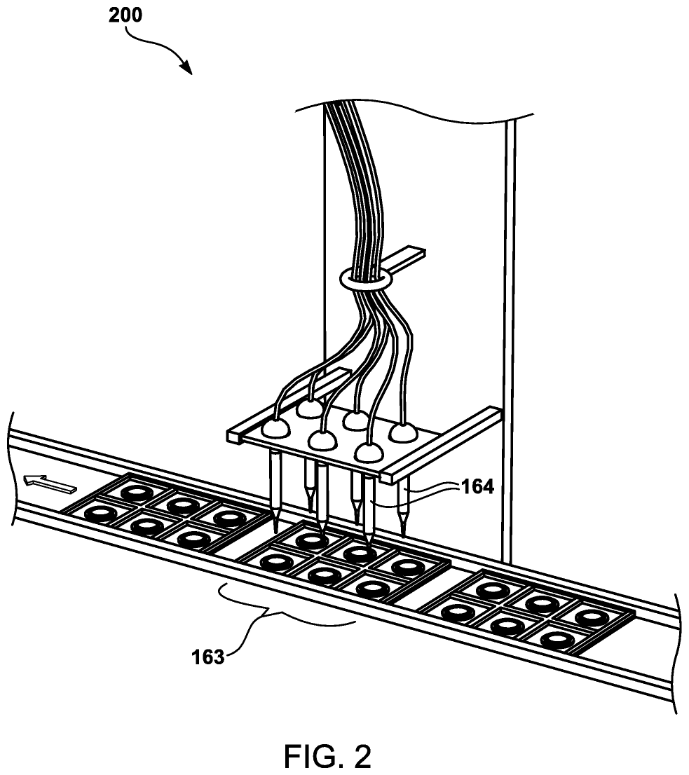

As an alternative, FIG. 2 illustrates a machine-operated fabrication configuration 200 to fabricate the aroma emission device, illustrated in FIG. 1C. For example, the machine-operated fabrication configuration 200 may have an automated dropper machine that emits droplets of the terpene solution into multiple second portions 163 of the aroma emission device. The machine-operated configuration 200 may also have machinery that adheres multiple first portions 162 to the second portions 163. As a result, the aroma emission device 161 may be mass-produced.

In yet another embodiment, an aroma emission device may be integrated into the structure of the cannabis storage container 105, illustrated in FIGS. 1A-1C. For example, the cap 106 of the cannabis storage container 105 may be manufactured such that the aroma storage device is built into the cap 106, or another portion of the cannabis storage container 105, rather than being a separate component that necessitates adherence.

[0031 ] Although the aroma emission device may be multi-purpose, it may also be single-purpose and used in conjunction with other single purpose devices within the cannabis storage container 105. For example, the cannabis storage container 105 may have both an aroma emission device and a humidity control device, which are distinctly adhered to inner portions of the cannabis storage container 105.

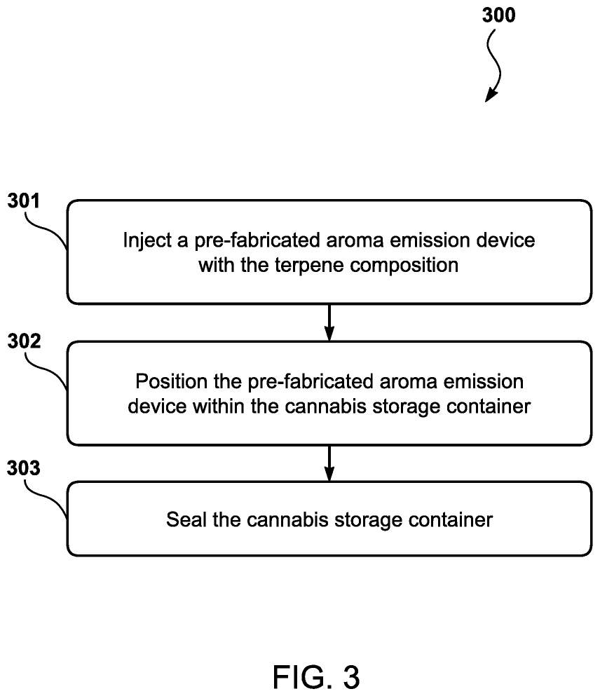

Finally, FIG. 3 illustrates a process for 300 for infusing a cannabis storage container with a terpene composition. At a process block 301, the process 300 injects the pre-fabricated aroma emission device 102 illustrated in FIG. 1A, or the alternative pre-fabricated aroma emission device 151 illustrated in FIG. 1B, with the terpene composition. The pre-fabricated aroma emission device 102 has one or more pores 104, or the pre-fabricated aroma emission device 151 has one or more pores 152, through which an aroma from the terpene composition is emitted without human activation after sealing of the cannabis storage container 105. Further, at a process block 302, the process 300 positions the pre-fabricated aroma emission device 102 within the cannabis storage container 105. At a process block 303, the process 300 seals the cannabis storage container 105.

Various types of apparatuses having different shapes (e.g., cylindrical, cubic, etc.) and sizes may be used for the cannabis storage container 105 and various aroma emission devices described herein. For example, the cannabis storage container 105 may vary in size from that of a jar to that of a bucket. Furthermore, as another example, the aroma emission device may vary in size from that of approximately a quarter-sized disk to a disk that almost entirely fits on the underside of a bucket cover.

Although the processes and apparatuses provided for herein are described with respect to storage of cannabis materials 107, they may also be utilized in conjunction with cannabis variants or other herbs.

It is understood that the apparatuses and processes described herein may also be applied in other types of apparatuses and processes. Those skilled in the art will appreciate that the various adaptations and modifications of the embodiments of the apparatuses and processes described herein may be configured without departing from the scope and spirit of the present apparatuses and processes. Therefore, it is to be understood that, within the scope of the appended claims, the present apparatuses and processes may be practiced other than as specifically described herein.

We claim:

1. A method for infusing a cannabis storage container with a terpene composition comprising:
injecting a pre-fabricated aroma emission device with the terpene composition, the pre-fabricated aroma emission device having one or more pores through which an aroma from the terpene composition is emitted;
positioning the pre-fabricated aroma emission device within the cannabis storage container; and
sealing the cannabis storage container,
wherein the pre-fabricated aroma emission device emits the aroma of the terpene composition through the one or more pores without human activation after said sealing of the cannabis storage container.

2. The method of claim 1, further comprising selecting the terpene composition according to a predetermined dilution threshold for cannabis odor emission.

3. The method of claim 1, further comprising validating compliance of the emission of the aroma, by the pre-fabricated aroma emission device, with the predetermined dilution threshold for cannabis odor emission prior to said sealing.

4. The method of claim 1, wherein the pre-fabricated aroma emission device is a geometrically shaped device that is adhered to a portion of the cannabis storage container, the portion being within the cannabis storage container.

5. The method of claim 4, wherein the portion is an underside of a cap for the cannabis storage container.

6. The method of claim 4, wherein the portion is a wall of the cannabis storage container.

7. The method of claim 1, wherein the pre-fabricated aroma emission device is integrated within a portion of the cannabis storage container.

8. The method of claim 1, wherein the pre-fabricated aroma emission device is a pouch.

9. The method of claim 1, wherein the pre-fabricated aroma emission device is a single-purpose aroma emission device that only performs the functionality of emitting the aroma without performing any additional functionality.

10. The method of claim 1, wherein the pre-fabricated aroma emission device is a multi-purpose aroma emission device that is configured, during fabrication of the pre-fabricated aroma emission device, to perform additional functionality, other than aroma emission.

11. The method of claim 1, wherein the terpene composition comprises a terpene blend comprising a plurality of naturally derived non-cannabis terpene isolates, a plurality of essential oils, and a plurality of flavor enhancers.

12. The method of claim 11, wherein the plurality of naturally derived non-cannabis terpene isolates are food grade.

13. The method of claim 11, wherein the plurality of flavor enhancers is selected from the group consisting of: an ester and an aldehyde.

14. The method of claim 1, wherein the terpene composition comprises a terpene blend of distinct terpenes extracted from one or more cannabis plants.

15. The method of claim 1, wherein said injection is effectuated via a syringe.

16. A method of manufacture of an aroma emission device, comprising:
positioning a terpene composition within a first portion of the aroma emission device; and
adhering a second portion of the aroma emission device to the first portion to seal the aroma emission device,
wherein the aroma emission device has one or more pores through which an aroma from the terpene composition is emitted,
wherein the aroma emission device is configured to be positioned within a cannabis storage container such that the aroma emission device emits the aroma of the terpene composition through the one or more pores without human activation after sealing of the cannabis storage container.

17. The method of claim 16, wherein the positioning of the terpene composition is effectuated via a dropper.

18. The method of claim 16, further comprising further comprising selecting the terpene composition according to a predetermined dilution threshold for cannabis odor emission.

19. The method of claim 16, wherein the pre-fabricated aroma emission device is a geometrically shaped device that is adhered to a portion of the cannabis storage container, the portion being within the cannabis storage container.

20. The method of claim 16, wherein the pre-fabricated aroma emission device is a pouch.
